(19) United States
(12) Patent Application Publication
(10) Pub. No.: US 2013/0035648 A1
(43) Pub. Date: Feb. 7, 2013

(54) TOTALLY IMPLANTABLE VENOUS DEVICE

(71) Applicants: Po-Jen Ko, Taipei City (TW); Ching-Yang Wu, Linkou Township (TW); Yun-Hen Liu, Guishan Shiang (TW)

(72) Inventors: Po-Jen Ko, Taipei City (TW); Ching-Yang Wu, Linkou Township (TW); Yun-Hen Liu, Guishan Shiang (TW)

(21) Appl. No.: 13/461,889

(22) Filed: May 2, 2012

Related U.S. Application Data
(63) Continuation-in-part of application No. 12/758,087, filed on Apr. 12, 2010, now abandoned.

(51) Int. Cl. A61M 5/00

(52) U.S. Cl. 604/288.01

(57) ABSTRACT

A totally implantable venous device includes a port, a catheter and a sleeving member. The port includes an arcuate housing having a recess on an inner surface, an opening communicating with the recess and having a flat mouth, an internal chamber, and a base formed of hard plastic and being capable of preventing a needle from being inserted therethrough. The catheter includes an enlarged fastening member at one end. The sleeve is complementarily mounted on a front portion of the enlarged fastening member. The sleeving member is complementarily mounted on the remaining portion of the enlarged fastening member. The recess and the opening form a through hole having an enlarged intermediate portion. The enlarged fastening member has a truncated trapezoidal shape. The sleeving member, the enlarged fastening member and a connector of a receiving seat in the port form a watertight structure at the through hole.

### FIELD OF INVENTION

The invention relates to totally implantable venous devices and, more particularly, to such totally implantable venous devices with improved characteristics including easy insertion, easy holding, and durability.

### DESCRIPTION OF RELATED ART

Conventional totally implantable venous devices are disadvantageous due to the following reasons: It is not easy to puncture, and only experienced doctors have higher success rate. Second, Catheter fracture may occur and the injection chamber and catheter have to be removed because of possible fatal complications. The implant seat or catheter is required to be removed from the patient if such occurs. Patients suffered from pain and these leads medical cost increased.

Conventionally, a totally implantable venous device comprises an injection chamber, a catheter, and a locking nut. In assembly, the catheter is inserted into the connection portion of the injection chamber, and, then, the locking nut is put on to fix the catheter. The connection portion of catheter is susceptible of fracture due to structural weaknesses. An integral type of totally implantable venous device is commercially available. However, implantation is more difficult if a small caliber of the blood vessel or a sharp angle between cephalic vein and axiliary vein are encountered.

Conventional totally implantable venous devices may be found to have mechanical failure because of more components. In other words, component simplification means improved performance.

Referring to FIG. 8, a conventional totally implantable venous device is shown in cross-section. A conventional totally implantable venous device consists of 3 components, including an injection chamber which contains a connecting portion, a catheter, and a locking nut. Because of the strength difference between the catheter and other components, catheter fracture may be encountered. In addition, the implant seat 1 of the port has a small outer surface 2 which limits the adjustment angle of the needle 6 and the metal wire 3 contained in the needle 6. Further, both the needle 6 and the metal wire 3 are difficult to insert through the outer surface 2, via the chamber 5, to the catheter 4. This may lead to some difficulties when the catheter is adjusted over the guide metal wire 3. Therefore, these disadvantages lead further structural improvement.

### SUMMARY OF THE INVENTION

It is therefore one object of the invention to provide a totally implantable venous device comprising a port comprising an arcuate housing and a base having an inner surface and an outer surface, with the inner surface defining an internal chamber, with the base formed of hard plastic and being capable of preventing a needle from being inserted therethrough; a through hole extending between the inner surface and the outer surface and having an enlarged intermediate portion between the inner and outer surfaces; an open receiving seat fastened in the port and comprising a tubular connector disposed in the through hole, the connector having a bulged central portion; a catheter comprising an enlarged fastening member at one end, wherein the enlarged fastening member having a front portion and a remaining portion; a sleeving member complementarily mounted on the remaining portion of the fastening member and having an inner end and an outer end; wherein the front portion of the enlarged fastening member has a truncated trapezoidal shape including a forward end and a rearward end, with the rearward end abutting with the inner end of the sleeving member; wherein the through hole has a shape complementary to the front portion of the fastening member and the sleeving member on the remaining portion of the fastening member such that a watertight structure is formed at the through hole when the sleeving member is put on the enlarged fastening member and together they are fitted in the through hole and securely put on the bulged central portion of the connector; and wherein the catheter and the enlarged fastening member are formed of the same material.

The above and other objects, features and advantages of the invention will become apparent from the following detailed description taken with the accompanying drawings.

### BRIEF DESCRIPTION OF THE DRAWINGS

FIG. 1 is an exploded view of a preferred embodiment of a totally implantable venous device according to the invention;

FIG. 2 is a cross-sectional exploded view of the assembled implant seat;

FIG. 3 is a perspective view of the assembled implant seat;

FIG. 4 is a cross-sectional view of the catheter with a metallic guide wire inserted thereinto;

FIG. 5 is an environmental view of the implant seat placed upon the body of a patient;

FIG. 6 schematically depicts a section of the metallic guide wire;

FIG. 7 is a perspective view of a puncture sheath of the invention; and

FIG. 8 is a cross-sectional view of a conventional totally implantable venous device.

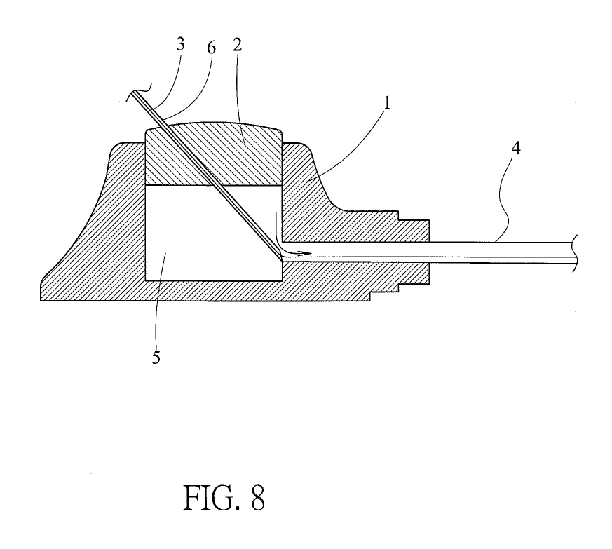

### DETAILED DESCRIPTION OF THE INVENTION

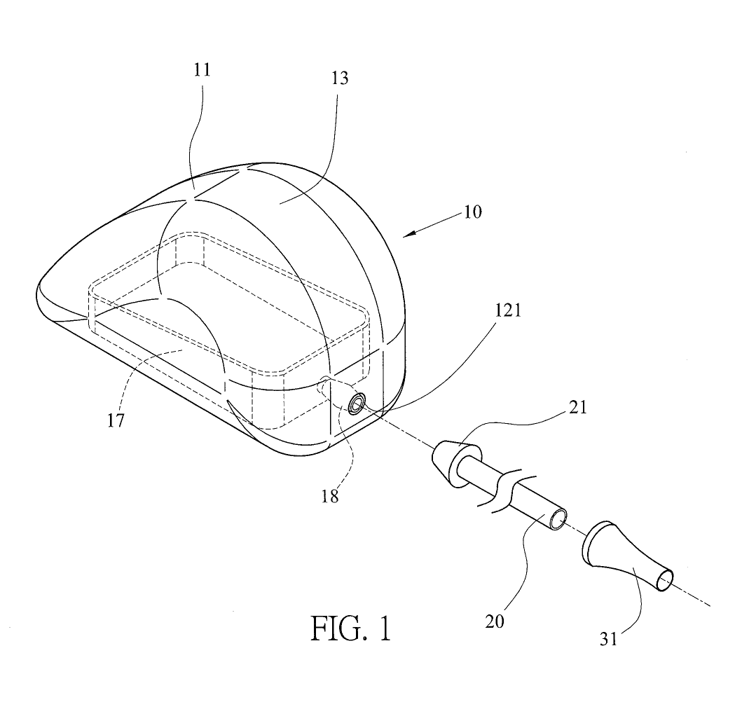

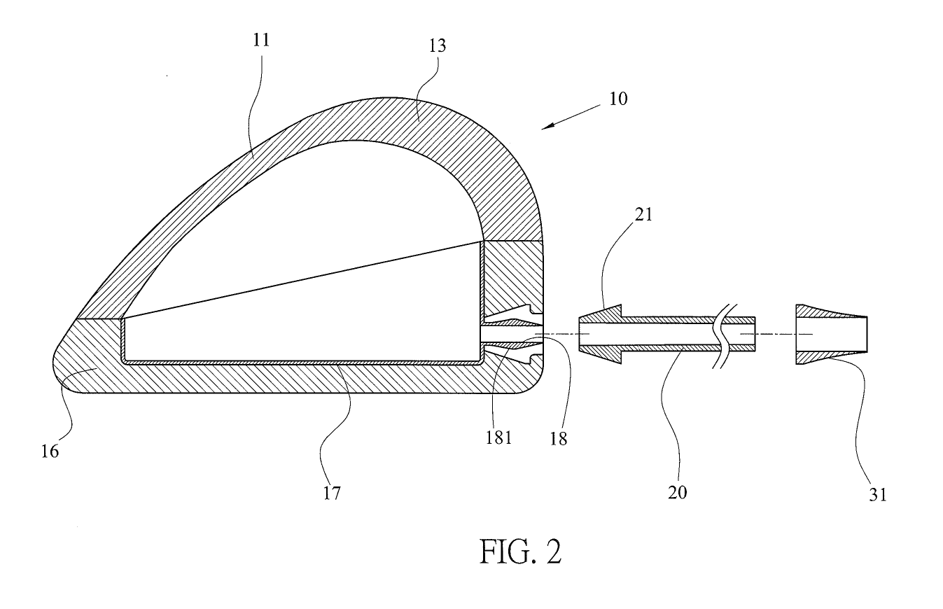

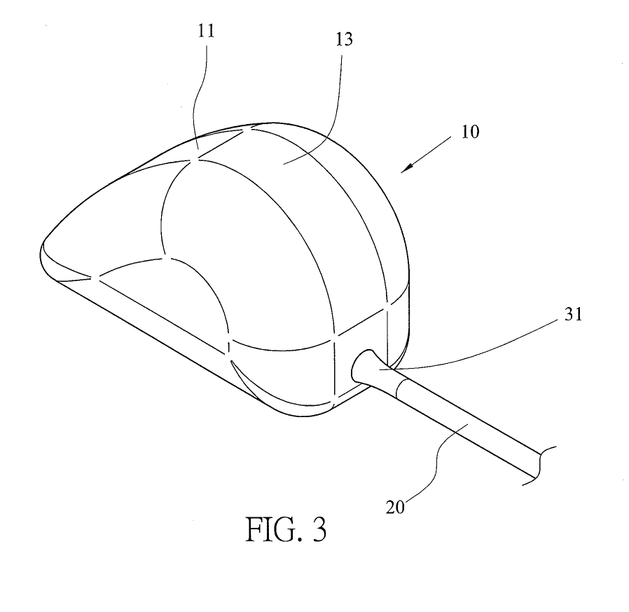

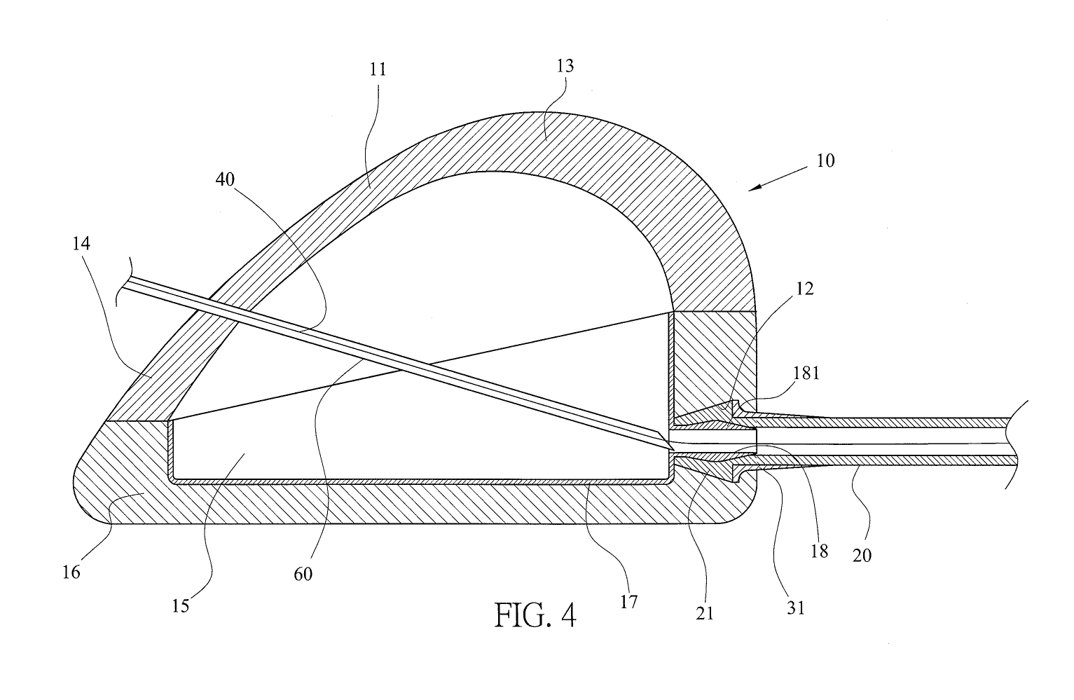

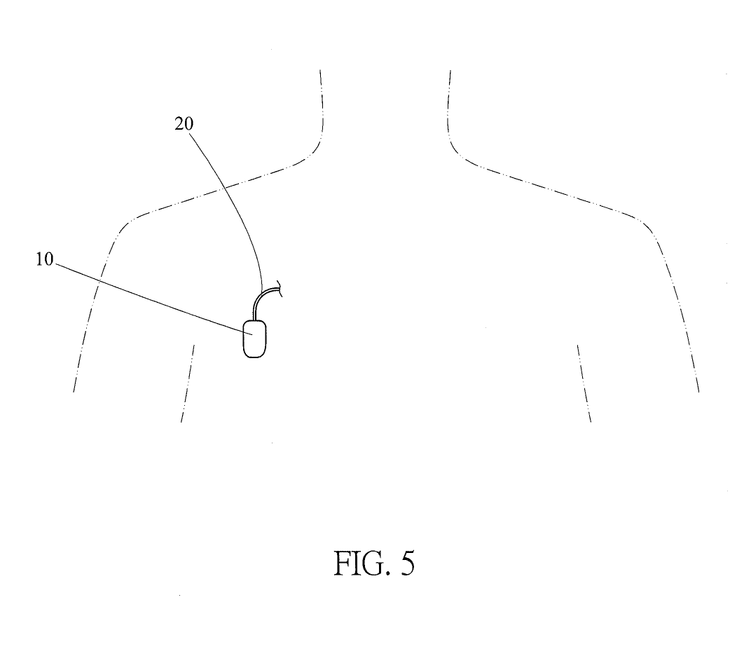

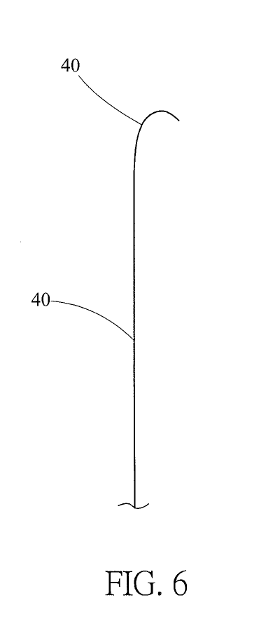

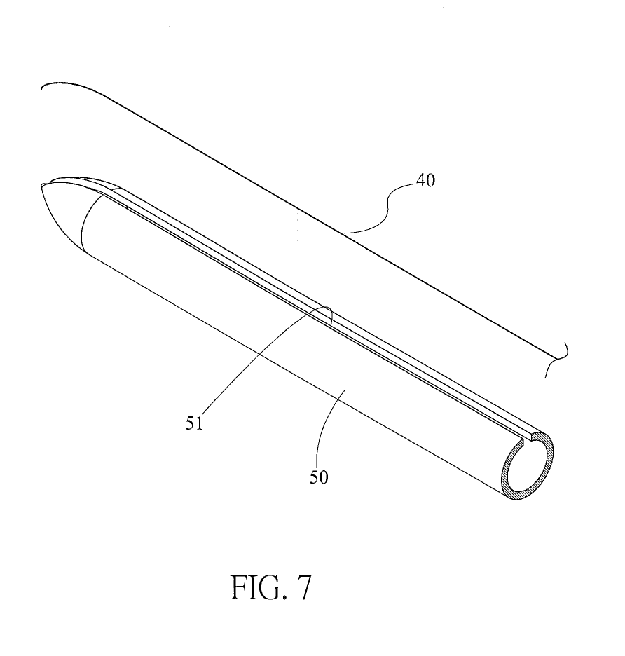

Referring to FIGS. 1 to 7, a totally implantable venous device in accordance with a preferred embodiment of the invention comprises the following components as discussed in detail below.

A hollow port 10 has an ergonomic shape for ease of holding by hand. The port 10 has a housing 11 including an arcuate surface 13 for allowing a needle to be inserted through from various angles, an internal chamber 15, and a base 16 formed of hard plastics. The base 16 is structurally strong to prevent a needle from being inserted therethrough.

A recess is formed on an inner surface of an upward extending frame of the base 16, and an opening 121 having a narrow flat mouth is formed on an outer surface of an upward extending frame of the base 16. The recess and the opening 121 together form a hole 12 through the upward extending frame of the base 16.

A receiving seat 17 having an open top is fastened in the chamber 15. A tubular connector 18 has one end integrally formed with the receiving seat 17 and the other end proximate to the mouth of the opening 121. The connector 18 has a bulged central portion 181.

A catheter 20 comprises an enlarged head 21 of a truncated trapezoidal shape at one end, wherein the enlarged head 21 having a front portion and a rear portion. An independent sleeving member 31 having a flare end and put on the rear portion of the enlarged head 21 in the opening 121. The sleeving member 31 and the enlarged head 21 together form a watertight structure at the through hole 12 and the opening 121. The enlarged head 21 can be flexibly compressed to fit in the connector 18 and releasably secured onto the bulged central portion 181. The enlarged head 21, the connector 18, and the sleeving member 31 together form a watertight structure.

Preferably, the sleeving member 31 can withstand a pressure in the range of 20 and 30 Bar. Still preferably, an upper portion 14 of the port 10 is formed of the same silicone as the catheter 20. Advantageously, the flat mouth of the opening 121 can reduce friction of the inserted catheter 20 and facilitate insertion.

In assembly, the sleeving member 31 is put on the rear portion of the enlarged head 21. The large intermediate portion of the through hole consisting of the through hole 12 and the opening 121 can fasten the catheter 20 after assembly. The sleeving member 31 is put on the enlarged head 21 which is in turn put on the bulged central portion 181 of the connector 18. As a result, the enlarged head 21 and the sleeving member 31 together are fastened in the opening 121.

Further, the catheter 20 and its enlarged head 21 are formed of the same material, and, together, they are not susceptible of breaking. Moreover, the arcuate surface 13 of the port 10 facilitates holding by hand in use. The receiving seat 17 is made of metal resistant to magnetic field (e.g., titanium alloy) or hard plastic. Further, the receiving seat 17 is resistant to needle insertion and further increases the structural strength of the port 10.

As a comparison, conventional totally implantable venous devices have rectangular bodies with four edges in contact with the skin, i.e., frictional contact. This undesirably may cause erosion of overlying skin. This is particularly true to cancer patients. Further, exposed implant seats are required to be removed or re-implanted to a new site. In this invention, the specific arcuate shape leads to less pressure and less friction between the implant seat and overlying skin.

Both totally implantable venous devices of the prior art and the invention are secured onto the chest. The implant seat of the invention has its port 10 being different from that of the prior art. As described above, the port 10 has an arcuate housing 11 which has the advantages of reducing friction with the skin and easy holding by hand.

It is envisaged by the invention that the receiving seat 17 is fastened in the port 10 with the connector 18 disposed in the through hole 12. The sleeving member 31 is put on the enlarged head 21 which is in turn put on the bulged central portion 181 of the connector 18. As a result, the enlarged head 21 and the sleeving member 31 together are fastened in the the through hole 12 to form a watertight structure. The receiving seat 17 is resistant to needle insertion and further increases the structural strength of the port 10 for withstanding pressure exerted by liquid contained in the port 10.

It is noted that the enlarged head 21 and the sleeving member 31 are together mounted in the through hole 12. This has the advantage of preventing the enlarged head 21 from being broken due to no protective means as experienced by the enlarged head of the conventional port. Further, the bulged central portion 181 can reduce stress generated by the assembled enlarged head 21 and the sleeving member 31. As a result, the structural strength of the port 10 is greatly increased.

The totally implantable venous device of the invention may cooperate with a metallic guide wire 40 which has a layer formed thereon. The metallic guide wire 40 may further cooperate with a hollow needle 60, i.e., passing through the needle 60. The needle 60 may be inserted into the port 10 to contact the catheter 20 in use.

Preferably, the metallic guide wire 40 has a length of about 50 cm.

The puncture sheath 50 has a longitudinal slit 51. The puncture sheath 50 can be utilized for small vessel cannulation. When a small caliber of a vessel is encountered, the metallic guide wire 40 is used to cannulate the vessel, and the puncture sheath 50 is mounted via the longitudinal slit 51 to create a subcutaneous tunnel. After the subcutaneous tunnel is created, the catheter 20 is advanced over-the-wire to the adequate position under intra-operative fluoroscopy. Finally, the catheter 20 is connected to the port 10.

The puncture sheath 50 is particularly useful for catheter implantation if implanted within a small caliber vessel or a large bent angle.

Conventional metallic guide wires are spiral. Portions of the metallic guide wires may be left within the body if there is no surrounding layer. Removal of a metallic guide wire 40 left within the body is difficult. Advantageously, the metallic guide wire 40 of the invention has a smooth surrounding layer. Hence, the metallic guide wire 40 of the invention is not susceptible of being left within the body after being pulled from the blood vessel. Further, it is easy to remove dirt from the metallic guide wire 40 of the invention. The conventional metal wire has a J-shaped end. Hence, it may jam within the chamber 15 of the port 1 after insertion, i.e., is difficult being removed. As an improvement, the metallic guide wire 40 of the invention has a slightly bent end 41. Hence, it is easy to remove from the chamber 15 of the port 1 after insertion.

The arcuate housing 11 of the port 10 of the invention can increase the injection area. Hence, it is easy to adjust angles of the cooperating metallic guide wire 40 and the needle 60. Further, it is easy to insert the metallic guide wire 40 through the chamber 15 of the port 10 to contact the catheter 20. Moreover, the metallic guide wire 40 may be inserted through the catheter 20 out of the port 10.

On the contrary, the conventional implant seat has a small injection area. Hence, the adjustment angle of the metal wire is limited. It is thus difficult to insert the metal wire through the chamber of the port and out of the port.

The metallic guide wire 40 of the invention has a slightly bent end 41. In comparison, the typical metal wire has a J-shaped end. Hence, the metallic guide wire of the invention is easy to use.

The portion connecting the catheter 20 to the port 10 is on an inner surface of the port 10. Hence, it is not easy to bend or even break the catheter 20. On the contrary, the conventional connection point is disposed externally of the port. Hence, it is susceptible of breaking. The catheter 20 and its enlarged head 21 are formed integrally. On the contrary, the conventional catheter and its head are separate members. The upper portion 14 of the port 10 is formed of the same silicone as the catheter 20. Moreover, the connecting portion of the port 10 and the catheter 20 are thin to allow vibration of the catheter 20.

The port 10 of the invention is ergonomic in shape. Hence, it is easy to hold the port 10. Further, its arcuate surface 13 facilitates needle insertion. Furthermore, its insertion angle can be adjusted easily. In addition, the arcuate port 10 of the invention has less friction with the skin.

While the invention herein disclosed has been described by means of specific embodiments, numerous modifications and variations could be made thereto by those skilled in the art without departing from the scope and spirit of the invention set forth in the claims.

What is claimed is:

1. A totally implantable venous device comprising:
a port comprising an arcuate housing and a base having an inner surface and an outer surface, with the inner surface defining an internal chamber, with the base formed of hard plastic and being capable of preventing a needle from being inserted therethrough;
a through hole extending between the inner surface and the outer surface and having an enlarged intermediate portion between the inner and outer surfaces;
an open receiving seat fastened in the port and comprising a tubular connector disposed in the through hole, the connector having a bulged central portion;
a catheter comprising an enlarged fastening member at one end, wherein the enlarged fastening member having a front portion and a remaining portion;
a sleeving member complementarily mounted on the remaining portion of the fastening member and having an inner end and an outer end;
wherein the front portion of the enlarged fastening member has a truncated trapezoidal shape including a forward end and a rearward end, with the rearward end abutting with the inner end of the sleeving member;
wherein the through hole has a shape complementary to the front portion of the enlarged fastening member and the sleeving member on the remaining portion of the fastening member such that a watertight structure is formed at the through hole when the sleeving member is put on the enlarged fastening member and together they are fitted in the through hole and securely put on the bulged central portion of the connector; and
wherein the catheter and the enlarged fastening member are formed of the same material.

2. The totally implantable venous device of claim 1, further comprising an elongated puncture sheath having a longitudinal slit for guiding insertion of the metallic guide wire.

3. The totally implantable venous device of claim 1, wherein the sleeving member has a truncated trapezoid shape with the inner end having a larger radial extent than the outer end.

4. The totally implantable venous device of claim 1, further comprising a metallic guide wire contained in a hollow needle insertable through the arcuate housing to contact the fastening member.

5. The totally implantable venous device of claim 1, wherein the receiving seat is made of metal resistant to magnetic field.

6. The totally implantable venous device of claim 5, wherein the metal is a titanium alloy.

7. The totally implantable venous device of claim 1, wherein the receiving seat is made of hard plastic.
